(19) United States
(12) Patent Application Publication
(10) Pub. No.: US 2012/0065504 A1
(43) Pub. Date: Mar. 15, 2012

(54) Method for measuring the viscoelastic properties of biological tissue employing an ultrasonic transducer

(75) Inventors: Laurent Sandrin, L'Hay-Les-Roses (FR); Veronique Miette, Villejuif (FR)

(73) Assignee: ECHOSENS SA, Paris (FR)

(21) Appl. No.: 12/308,418

(22) PCT Filed: Jun. 15, 2007
(86) PCT No.: PCT/FR2007/001002

§ 371 Date: Jun. 23, 2010

(30) Foreign Application Priority Data
Jun. 15, 2006 (FR) 0605342

(51) Int. Cl. A61B 8/00

(52) U.S. Cl. 600/438

(57) ABSTRACT

The present invention relates to a Method of measuring the viscoelastic properties of biological tissues employing an ultrasonic transducer equipped with elements converting the ultrasonic waves reflected by these biological tissues into electrical signals, different elements being grouped to form sub-apertures such that the acquisition of electrical signals from the elements of the same sub-aperture is carried out simultaneously, each of these sub-apertures being intercepted by an ultrasonic wave propagation axis at an acoustic center (Ca).

In conformance with the invention, such a method is characterized in that one and the same element belongs to at least two different sub-apertures and in that an acoustic center is surrounded by at least three other unaligned acoustic centers.

Measuring the viscoelastic properties, subsequently referred to as VP, of biological tissues allows the diagnosis, screening or monitoring of treatments related to, for example, organs such as the liver, skin or blood vessels.

### BACKGROUND

In order to carry out a non-invasive measurement respecting the integrity of a relevant organ, using a device emitting a low-frequency shearing wave in the biological tissues of this organ and then measuring, by means of ultrasonic signal acquisitions, the response of the biological tissue to this shearing wave is known. Such a method is described, for example, in patent application FR 2869521 filed on 3 May 2004 in the name of the Echosens Corporation.

A method for measuring the VP of biological tissues utilizing a device included in one of the three categories described below is known:

A first device category presents a transducer comprised of a single element converting the ultrasound waves reflected by the relevant tissues into electrical signals. In this case, such a transducer employs a single ultrasonic beam, which does not allow the VP of heterogeneous organs to be measured.

The use of such a device is thus limited to an average VP measurement for the entire organ, such an average measurement not allowing local VP measurements to be made in view of detecting, for example, a localized pathology in this organ.

A second device category presents a transducer comprising an alignment of elements converting the ultrasonic waves reflected by the relevant tissues into electrical signals.

In this case, such a transducer only receives ultrasonic waves reflected relative to a two-dimensional plane. Such being the case, obtaining the VP's from a plane of biological tissue necessitates three-dimensional volume data, particularly in elevation, that is, according to a direction orthogonal to the relevant plane.

Because of this, the use of such a device does not allow the VP's of the relevant tissue to be quantitatively measured. In other words, such a device only provides local qualitative information that is subjected to artifacts due to the approximation carried out by disregarding variations in tissue deformations produced in elevation.

Finally, a third device category, described in patent application FR 2869521, previously cited, uses a transducer comprising four unaligned circular elements converting the ultrasonic waves reflected by the relevant tissues into electrical signals.

This type of device aims to, in particular, provide a quantitative measurement of the VP's of biological tissues. But it presents the disadvantage of using elements whose large dimensions are strictly required by the desired ultrasonic beam characteristics. By way of example, in the liver, with a 3.5 Mhz central frequency ultrasonic transducer, this necessitates elements with a diameter of 7 mm to be able to carry out measurements at depths of between 20 and 80 mm.

### SUMMARY OF THE INVENTION

The invention resolves at least one of the previously indicated problems by providing a method of measuring the VP's of biological tissue that allows quantitative measurements of local VP's to be made with satisfactory resolution.

It is on object of the present invention to provide a method of measuring the VP of biological tissues employing an ultrasonic transducer equipped with elements converting ultrasonic waves reflected by these biological tissues into electrical signals, various elements being grouped together to form sub-apertures such that the acquisition of electrical signals coming from the elements of any one sub-aperture is carried out simultaneously, each of these sub-apertures being intercepted by an ultrasonic wave propagation axis at an acoustic center, characterized in that at least one and the same element belongs to at least two different sub-apertures and in that an acoustic center is surrounded by at least three other unaligned acoustic centers.

It should be noted that an ultrasonic wave propagation axis corresponds to the axis at which the distribution of energy is maximum.

Such a method presents numerous advantages. Because of this, the use of at least one common element with different sub-apertures allows the distance between the acoustic centers of different sub-apertures to be reduced without reducing the ultrasonic wave transmitting and receiving surface, which allows VP measurements to be made with increased resolution.

In addition, the fact that the acoustic center of a sub-aperture is surrounded by at least three unaligned acoustic centers allows the necessary volume data to be obtained to be able to calculate the local VP's, as detailed subsequently.

Lastly, the joint employment of the two layouts indicated above allows a smaller number of elements transforming ultrasonic waves into electrical signals to be used while having improved resolution with relation to the prior art for making local VP measurements.

Consequently, the cost and complexity of the VP measurement method in conformance with the invention are reduced while this same method may make local measurements, even quantitative measurements, of the VP's of tissues from an organ with a resolution sufficient for measuring localized VP's capable of identifying, for example, a tumor in an organ.

In one embodiment, the method comprises the step of using different sub-apertures simultaneously, for example, by using the same element in sub-apertures used simultaneously.

The simultaneous use of sub-apertures allows a faster rate of acquisition of ultrasonic data to be obtained. In fact, the use of at least one common element with different sub-apertures is comparable to two simultaneous for this same element that enables a higher rate to be obtained than that which would be possible if the signals from each sub-aperture were formed sequentially.

The formation of channels for a given sub-aperture corresponds to the summation, with or without time lags (time delay law), of signals from different elements constituting this sub-aperture.

This summation may be carried out according to several methods; by way of example the summation of electrical analog signals from different elements, the summation in an electronic component after digitization and software summation in a computer program may be cited.

In fact, sequential electronic scanning of sub-apertures is replaced by at least one parallel, that is to say, simultaneous, acquisition for these sub-apertures.

In one embodiment, the method comprises the step of driving tissues in movement; this movement may be carried out manually or automatically.

In one embodiment, the method comprises the step of forming sub-apertures such that the acoustic centers of these sub-apertures form a grid presenting a triangular mesh, for example equilateral.

According to one embodiment, the method comprises the step of forming sub-apertures such that a sub-aperture is entirely defined by the surface of other sub-apertures.

In one embodiment, the method comprises the additional step of forming sub-apertures such that an acoustic center is surrounded by six equidistant acoustic centers.

The invention also relates to a device for measuring the VP of biological tissues equipped with an ultrasonic transducer comprising elements that convert the ultrasonic waves reflected by these biological tissues into electrical signals, characterized in that the elements are situated at a distance, measured between their centers, of between 0.5 and 5 mm, preferentially between 2 and 5 mm.

Such a distance between transducer elements is obtained thanks to the employment of a method in conformance with one of the previously described embodiments. Also, such a device presents the advantage of allowing the VP to be measured with satisfactory resolution at a low cost considering the smaller number of elements required for implementation of the invention.

According to one embodiment, the device comprises means for simultaneously acquiring electrical signals received by a plurality of elements grouped in one sub-aperture and means for forming electrical signal transmission channels corresponding to several sub-apertures simultaneously presenting at least one common element.

In one embodiment, the device comprises means so that the center of at least one sub-aperture is surrounded by at least three unaligned acoustic centers.

According to one embodiment, the device comprises at least 19 hexagonal elements or at least 24 equilateral triangular elements.

In one embodiment, the device comprises elements having the shape of a polygon, for example, a hexagon, a square, a diamond or a triangle, or a circle.

The invention also relates to a probe equipped with a device in conformance with one of the previous embodiments as well as a system equipped with a device in conformance with one of the previous embodiments, this system in addition comprising means to carry out ultrasound hyperthermia treatment or to drive tissues in movement.

Lastly, the invention relates to data from a method, a device, a probe or a system in conformance with one of the previous embodiments.

### BRIEF DESCRIPTION OF THE DRAWINGS

Other characteristics and advantages of the invention will appear in light of the description of an embodiment of the invention below, by way of illustrative and non-limiting example, with reference to the attached figures in which:

FIG. 1 is a diagram of a device comprising a transducer in conformance with the invention,

FIG. 2 illustrates the geometric parameters used to measure the VP of a biological tissue, and

FIGS. 3, 4 and 5 illustrate various implementations of a method in conformance with the invention.

### DETAILED DESCRIPTION

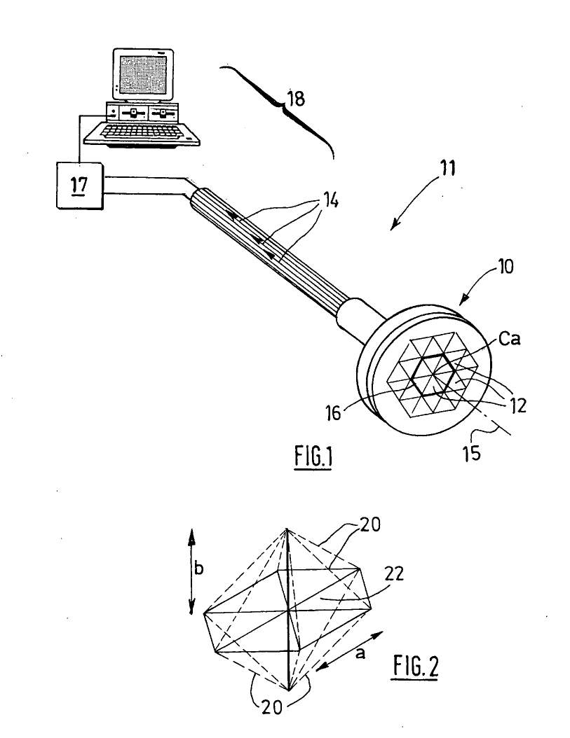

A method of measuring the VP of biological tissues in conformance with the invention employs a probe 11 (FIG. 1) equipped with an ultrasonic transducer 10 comprising elements 12 that convert the ultrasonic waves reflected by the biological tissues into electrical signals 14.

These electrical signals 14 are representative of the echogenicity of tissues in relation to ultrasonic waves. Thus, a tissue is called “hyperechogenic” when it strongly reflects ultrasonic waves while it is called “hypoechogenic” when it weakly reflects ultrasonic waves.

During the implementation of a method in conformance with the invention, different elements 12 form groups known as sub-apertures such that the acquisition of signals 14 issued from elements 12 from the same sub-aperture 16 is carried out simultaneously.

Thus, during this acquisition, the total acquisition surface of the sub-aperture 16 is the sum of the surfaces of its elements 12.

At this stage, it should be noted that the transducer 10 may also be used to transmit ultrasonic waves intended to be reflected by the relevant biological tissues. In this case, these elements 12 may be grouped into a transmission sub-aperture while different sub-apertures 16 may transmit simultaneously.

In addition, a sub-aperture 16 is characterized by an axis 15 along which the beam of ultrasonic waves transmitted or received by this sub-aperture 16 is propagated, this axis 15 intercepting the sub-aperture in a point known as the acoustic center Ca. For reasons of clarity, only axis 15, sub-aperture 16 and center Ca are represented in FIG. 1.

In conformance with the invention, while measuring the VP of a biological tissue, the method uses different sub-apertures 16 such that at least one and the same element 12 belongs to at least two different sub-apertures 16 while the acoustic center Ca of a sub-aperture 16 is surrounded by at least three other unaligned acoustic centers.

The use of a common element 12 with different sub-apertures allowing the distances between the acoustic centers of different sub-apertures to be reduced and resolution to be increased, this latter may be on the order of a millimeter, is obtained even though the method uses a smaller number of elements, typically fewer than thirty.

In this embodiment, considering the use of acquisitions made in parallel over several sub-apertures 16 simultaneously, the rate of acquisition of ultrasonic data is very fast in comparison with the rate that would be obtained with conventional sequential electronic screening, that is, using sub-apertures employed successively.

The rate thus obtained is uniquely limited by the ultrasonic wave propagation time and by the duration of the repetition echos. This rate, typically on the order of 4 KHz, and more generally between 100 Hz and 20 KHz, allows the volume deformations produced in the tissues by the propagation of the shearing wave from a single shearing excitation to be measured.

At this stage, it should be noted that this shearing excitation may be performed by using a vibrator external to the organ, an organic vibration generated by an organ from the body or a vibration triggered remotely, for example, by using the principle of radiation pressure.

Considering the very short propagation duration of the shearing wave, on the order of a hundred milliseconds, the acquisition of volume and local data may be done on mobile organs, for example the liver.

A system 18 for measuring the VP comprising probe 11 may comprise means 17 for simultaneously acquiring signals received by a plurality of elements grouped into a plurality of sub-apertures, during the ultrasonic wave reception phase, and means to form channels corresponding to the several sub-apertures used simultaneously.

Thus, such a device allows a method in conformance with the invention to be implemented with a high rate.

In addition, the fact that the acoustic center Ca of a sub-aperture 16 is surrounded by at least three unaligned acoustic centers allows the volume data necessary for being able to calculate local viscoelastic parameters such as the shear modulus, viscosity, Young's modulus, and Poisson's ratio to be obtained.

For example, by considering the elasticity, or Young's modulus noted E, this calculation may be made by using the operations indicated in the patent application FR 2869521 previously cited. In fact, the elasticity E of a tissue may be calculated from the following equation:

E=3ρVₛ²

Where ρ is the density of the medium and Vs represents the propagation speed of the shearing wave.

Assuming the medium is isotropic and linear, the shearing speed Vs verifies

$${V}_{s} = \sqrt{\frac{{\partial }^{2} u / \partial {t}^{2}}{Δ u}} .$$

Where u is the displacement, the deformation or speed of deformation measured according to a given direction and Δu is the Laplace operator of u.

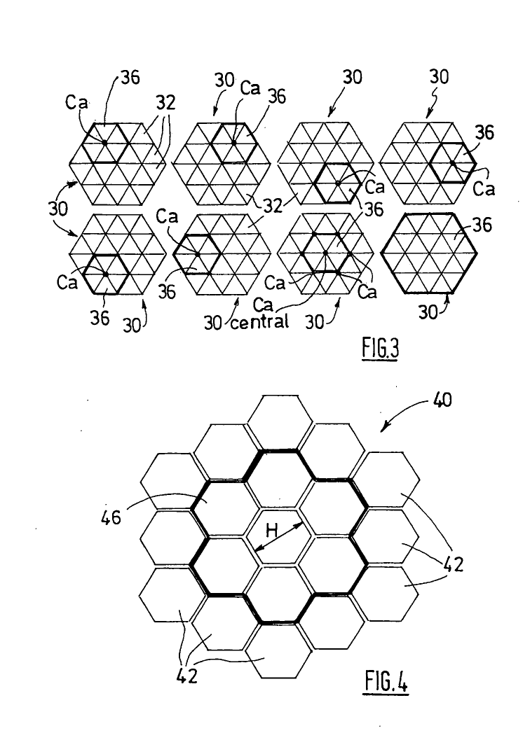

Obtaining the parameters of deformations along axes 20 (FIG. 2) situated in the center and apices of a hexagonal plane 22 allows the Laplace operator of the displacement u to be exactly calculated while, contrary to the elastographic techniques cited previously, no simplifying hypothesis regarding the expression of the Laplace operator is necessary, this expression being taken in its entirety.

By way of example, the discretization of the Laplace operator of u in a point i, the center of the hexagon, may be written according to the values of u at the apices j of the hexagon:

$${( Δ u )}^{i} = \frac{1}{\sqrt{3} {a}^{2}} {\sum }_{j = 1}^{6} \frac{2}{\sqrt{3}} ( {u}^{j} - {u}^{i} ) + \frac{1}{{b}^{2}} ( {u}^{z} + {u}^{- z} - 2 {u}^{i} ) .$$

Where a and b represent the lateral dimensions and uz and u−z are values of u in elevation with relation to the relevant hexagon 22.

The shearing speed may be obtained from the following equation:

$${V}_{s} = \sqrt{\frac{\frac{{u}^{i , t +} + {u}^{i , t -} - 2 {u}^{i , t}}{{T}^{2}}}{[ \frac{1}{\sqrt{3} {a}^{2}} {\sum }_{j = 1}^{6} \frac{2}{\sqrt{3}} ( {u}^{j , t} - {u}^{i , t} ) + \frac{1}{{b}^{2}} ( {u}^{z + , t} + {u}^{z - , t} - 2 {u}^{i , t} ) ]}} , \\ ∀ t ∈ [ {t}_{min} , {t}_{max} ]$$

Where tmin and tmax define the measurement period.

Thus it seems that the minimum resolution of a device depends on the distances between the acoustic centers of the sub-apertures, the resolution being all the higher as this distance is shorter.

Typically, this distance is less than 3 mm, more generally between 1 and 3 mm, with a transducer device proposed in this application that provides a satisfactory resolution of 1 mm.

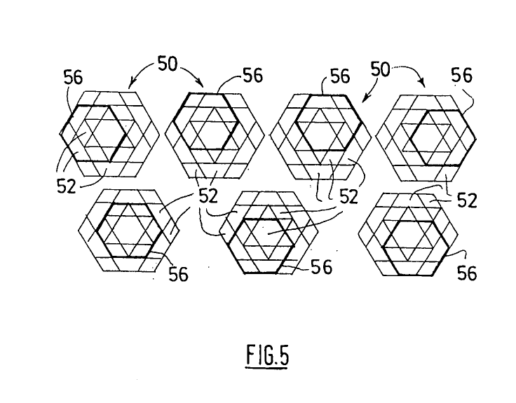

An embodiment of the invention is detailed below with the help of FIG. 3 that represents different sub-apertures 36, and their associated centers Ca, formed by elements 32 of a transducer 30, these different sub-apertures 36 being illustrated on different diagrams of the same transducer 30 for reasons of clarity.

Thus it is observed that, during acquisition of ultrasonic signals, the acoustic centers Ca of these different sub-apertures 36 may form a grid presenting a triangular mesh 22, for example an equilateral triangular mesh.

Such a triangular mesh presents the interest of limiting the distance between acoustic centers Ca at the distance from the side of the triangle formed by each element 32. In this embodiment, such a distance is 3 mm with elements operating at a central frequency of 3 MHz.

In this case, it seems that the sub-apertures 36 have a hexagonal shape wherein the sides have a length of 3 mm.

This transducer 30 allows VP to be measured at depths of between 10 and 90 mm, this depth being measured from the surface of the transducer.

When sub-aperture 36 is entirely surrounded or defined by a plurality of other sub-apertures 36, used simultaneously, the quantity of data relative to the relevant volume is increased such that the precision of the VP calculation is improved.

In this example, a central acoustic center Cacentral may be surrounded by six equidistant acoustic centers Ca.

The equidistance of the acoustic centers Ca simplifies the VP calculation by introducing symmetry in the discretization of the elastic wave propagation equation.

The transducer 30 illustrated comprises 24 equilateral triangular elements 32 and seven sub-apertures 36 comprised of six elements 32 while a sub-aperture 36 comprises the set of elements 32.

A probe equipped with such an ultrasonic transducer allows VP measurements to be made with a depth that depends on the central frequency of said transducer.

The implementation of a method in conformance with the invention, such as previously described, may be performed by using a device for measuring the VP of biological tissues equipped with an ultrasonic transducer comprising elements whose centers are situated at a shorter distance, for example between 0.1 and 5 mm and preferentially between 2 and 5 mm.

In addition, two sub-apertures used simultaneously may comprise at least one common element so as to increase the data acquisition speed and the temporal coherence of the data obtained while reducing the distance between the acoustic centers of the sub-apertures presenting the common element.

To enable volumetric analysis, the acoustic center Ca of at least one sub-aperture must be preferentially surrounded by at least three acoustic centers, unaligned between each other, corresponding to the sub-apertures used simultaneously.

In a second embodiment, such as described in FIG. 4, the transducer 40 comprises at least 19 hexagonal elements 42. In this case, one may use sub-apertures constituted of seven elements, such as the sub-aperture 46 represented.

In this embodiment, hexagonal elements 42 have a height H of two millimeters and are used with a central frequency of 3.5 MHz. In this case, the resolution obtained is on the order of a millimeter, this resolution being defined as the dimension of the smallest tissue volume measured.

According to a third embodiment (FIG. 5), elements 52 have variable shapes that nevertheless allow sub-apertures 56 with identical geometries, that is, hexagonal, to be formed.

In fact, the present invention is capable of having numerous variations. It may be implemented with elements of different shapes such as: polygons (for example hexagonal, square or diamond, triangle), or circular shapes, or combinations of elements with different shapes.

In addition, a device in conformance with the invention may be coupled or integrated with a larger-size system.

An example of this would be a system comprising a transducer performing ultrasonic hyperthermia treatment.

According to another example, a system comprising means to drive tissues in movement such that an ultrasonic transducer employs radiation pressure, the term from the English “remote palpation” or “acoustic radiation force.”

A last example is a system comprising means to drive tissues in movement employing an electromechanical vibrator.

Independently from the nature of the means implemented to drive the tissues in movement, synchronization is carried out between these means and the acquisition of ultrasonic data, this acquisition may comprise the storage of digital data obtained from electrical signals issued from the transducer and/or the processing of said data.

It should be noted that the previously mentioned transducers are ultrasonic transducers, that is, transducers converting electrical energy, respectively ultrasonic, into ultrasonic energy, respectively electrical.

Lastly, the invention is capable of being implemented according to different variations:

In a first variation, the pattern formed by the elements of a transducer in conformance with the invention, for example in FIG. 3, 4, 5 or 6 is repeated. Such a repetition may be carried out in one or more distinct directions.

In another variation, a first pattern in conformance with the invention is completed by the elements forming, for example, a second pattern.

What is claimed is:

1-13. (canceled)

14. A method of measuring viscoelastic properties of biological tissues employing an ultrasonic transducer equipped with elements converting the ultrasonic waves reflected by these biological tissues into electrical signals, comprising:
grouping different elements of the elements to form sub-apertures such that acquisition of the electrical signals from the elements of a same sub-aperture is carried out simultaneously, each of the sub-apertures being intercepted by an ultrasonic wave propagation axis at an acoustic center, at least one element belonging to at least two different sub-apertures and one of the acoustic centers being surrounded by at least three other unaligned acoustic centers of the acoustic centers.

15. The method according to claim 14 further comprising the step of using different sub-apertures simultaneously.

16. The method according to claim 15 further comprising the step of using the same element in the sub-apertures simultaneously.

17. The method according to claim 14 further comprising the step of driving the tissues in movement.

18. The method according to claim 14 further comprising the step of forming sub-apertures in such a way that the acoustic centers of the formed sub-apertures form a grid presenting a triangular mesh.

19. The method according to claim 18 wherein the triangular mesh is an equilateral triangular mesh.

20. The method according to claim 14 further comprising the step of forming sub-apertures in such a way that one of the sub-apertures is entirely defined by the surface of other sub-apertures.

21. The method according to claim 14 comprising the step of forming sub-apertures in such a way that the surrounded acoustic center is surrounded by six equidistant acoustic centers.

22. A device comprising:
means for measuring viscoelastic properties of biological tissues employing an ultrasonic transducer equipped with elements converting the ultrasonic waves reflected by these biological tissues into electrical signals, different elements of the elements being grouped to form sub-apertures such that the acquisition of electrical signals from the elements of a same sub-aperture is carried out simultaneously, each of the sub-apertures being intercepted by an ultrasonic wave propagation axis at an acoustic center, and
means so that at least one and the same element belongs to at least two different sub-apertures of the sub-apertures, one of the acoustic centers being surrounded by at least three other unaligned acoustic centers of the acoustic centers.

23. The device according to claim 22 further comprising means for simultaneously acquiring electrical signals received by a plurality of elements grouped together in a sub-aperture and means for carrying out the formation of channels corresponding to several simultaneous sub-apertures presenting at least one common element.

24. The device according to claim 22 wherein the elements include at least 19 hexagonal elements or at least 24 equilateral triangular elements.

25. The device according to claim 22 wherein the elements include elements having the shape of a polygon.

26. The device according to claim 22 wherein the polygon is a hexagon, square, diamond or triangle, or a circle.

27. A probe equipped with a device according to claim 22.

28. A system comprising: a device according to claim 22; and means for performing ultrasonic hyperthermia treatment or for driving tissues in movement.

29. A device comprising:
a measuring device measuring viscoelastic properties of biological tissues employing an ultrasonic transducer equipped with elements converting the ultrasonic waves reflected by these biological tissues into electrical signals, different elements of the elements being grouped to form sub-apertures such that the acquisition of electrical signals from the elements of a same sub-aperture is carried out simultaneously, each of the sub-apertures being intercepted by an ultrasonic wave propagation axis at an acoustic center, at least one and the same element belonging to at least two different sub-apertures of the sub-apertures, one of the acoustic centers being surrounded by at least three other unaligned acoustic centers of the acoustic centers.
